(19) United States
(12) Patent Application Publication
(10) Pub. No.: US 2022/0168090 A1
(43) Pub. Date: Jun. 2, 2022

(54) TUBULAR INDWELLING DEVICE

(71) Applicant: KAWASUMI LABORATORIES, INC., Saiki-Shi, Oita (JP)

(72) Inventor: Noriaki Shirahama, Bungo-ono-shi, Oita (JP)

(21) Appl. No.: 17/422,656

(22) PCT Filed: Jan. 31, 2020
(86) PCT No.: PCT/JP2020/003689

§ 371 Date: Feb. 9, 2022

(30) Foreign Application Priority Data
Feb. 4, 2019 (JP) 2019-017969

(51) Int. Cl. A61F 2/04

(52) U.S. Cl. CPC A61F 2/04; A61F 2002/041

(57) ABSTRACT

This tubular indwelling device 1, which is placed in a living body lumen (bile duct) to define a tubular flow path, comprises: a tubular body part 2; and a valve part 3 that is provided on a downstream end part 2a of the tubular body part 2 in a flow direction of a fluid (bile) flowing through the living body lumen and has an outflow port 23 from which the fluid flows out. The valve part 3 further comprises, for example, a “V”-shaped guide part 30 that guides the insertion of a jig 200 (not illustrated) into the outflow port 23.

### TECHNICAL FIELD

The present invention relates to a tubular indwelling device.

### BACKGROUND ART

Conventionally, there have been known tubular indwelling devices that are placed in a living body lumen such as a blood vessel and a digestive tract. This type of tubular indwelling device generally has a tubular shape and includes a framework part capable of expanding and contracting in a radial direction and a membrane part arranged along the framework part. In addition, for example, in some tubular indwelling devices used for treating stenosis or occlusion of a bile duct, a cylindrical projection part that cylindrically protrudes from one end of a body part is formed from a film body (e.g. see Patent Document 1).

### PRIOR ART DOCUMENT

### Patent Document

Patent Document 1: Japanese Unexamined Patent Application Publication No. H7-275369

### SUMMARY OF THE INVENTION

### Technical Problem

Incidentally, there are a case of using an endoscope for confirming a state of a tubular indwelling device placed in a living body lumen, a state of a lumen inner face behind the tubular indwelling device in the living body lumen, and the like, and a case of using a treatment device for treating a part behind the tubular indwelling device in the living body lumen. However, when a film body of a cylindrical projection part is in close contact with an opening to close the opening, it is difficult to insert the endoscope or treatment device into an inside of the tubular indwelling device, resulting in a problem that observation or treatment cannot be easily conducted. On the other hand, for properly suppressing reverse flow of foreign substances from a duodenum to a bile duct, it is desirable to close the opening of the cylindrical projection part when a bile is not released from a gallbladder. The aforementioned problem may occur not only in the tubular indwelling device for bile duct but also in a tubular indwelling device having a check valve-like function (hereinafter, referred to as “valve function”) in the same manner.

An object of the present invention is to provide a tubular indwelling device capable of easily inserting a jig into the inside of the tubular indwelling device having a valve part.

### Solution to Problem

The tubular indwelling device according to the present invention is placed in a living body lumen to define a tubular flow path, and includes

a tubular body part, and

a valve part provided on a downstream end part of the tubular body part in a flow direction of a fluid flowing through the living body lumen and having an outflow port from which the fluid flows out, wherein

the valve part further has a guide part for guiding insertion of a jig into the outflow port.

### Advantageous Effect of the Invention

According to the present invention, a jig can be easily inserted into an inside of a tubular indwelling device having a valve part.

### BRIEF DESCRIPTION OF THE DRAWINGS

FIG. 1 is a perspective view illustrating an end part of a tubular indwelling device according to an embodiment of the present invention.

FIG. 2 is a plan view of the end part of the tubular indwelling device in FIG. 1.

FIG. 3 (a) is a diagram for explaining insertion of a jig into a valve part of the tubular indwelling device in FIG. 1.

FIG. 3 (b) is a diagram for explaining insertion of the jig into the valve part of the tubular indwelling device in FIG. 1.

FIG. 3 (c) is a diagram for explaining insertion of the jig into the valve part of the tubular indwelling device in FIG. 1.

FIG. 4 is a perspective view illustrating an end part of a tubular indwelling device according to another modification example of the embodiment of the present invention.

### DESCRIPTION OF THE EMBODIMENT

Hereinafter, the embodiment of the present invention will be explained in detail with reference to the figures.

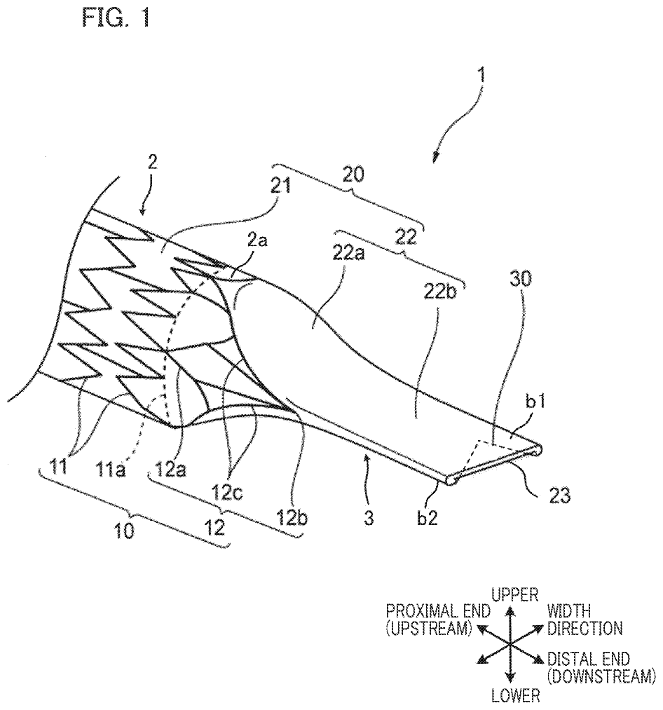

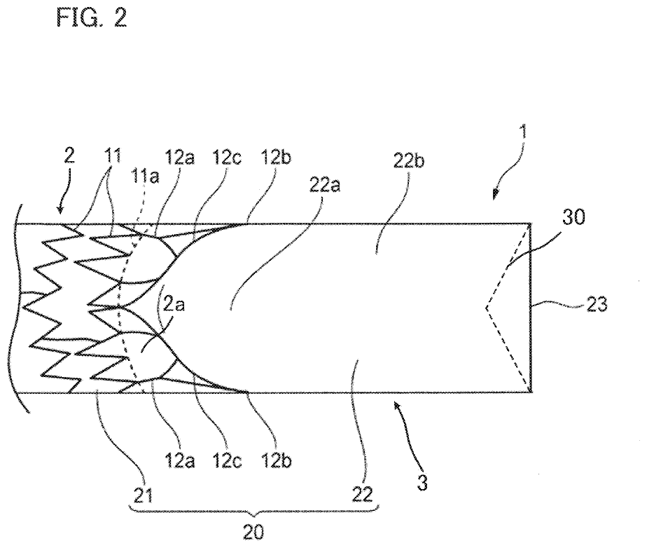

FIG. 1 is a perspective view illustrating a downstream end part 2a of a tubular indwelling device 1 according to the embodiment of the present invention, and FIG. 2 is a plan view of the downstream end part 2a of the tubular indwelling device 1. Note that, in the following description, a longitudinal direction of the tubular indwelling device 1 is defined as a “tube axis direction”, a direction orthogonal to the “tube axis direction” is defined as a “width direction”, and a direction orthogonal to the “tube axis direction” and the “width direction” is defined as a “vertical direction”. In addition, one end side (gallbladder side) in the “tube axis direction” in a state that the tubular indwelling device 1 is placed is defined as a “proximal end side”, and the other end side (duodenum side) is defined as a “distal end side”.

The tubular indwelling device 1 is placed in e.g. a bile duct (living body lumen) and widens a lesion site such as an occluded site or a stenosis site of the bile duct outward in a radial direction to treat the lesion site (generally, referred to as a bile duct stent). At this time, the tubular indwelling device 1 is placed such that the proximal end side and the distal end side are oriented to the gallbladder side and the duodenum side respectively, so that the proximal end side is the upstream side and the distal end side is the downstream side in a bile flow direction.

As illustrated in FIG. 1 and FIG. 2, in the tubular indwelling device 1, a valve part 3 having an outflow port 23 (described later) is provided on e.g. the downstream end part 2a of the tubular body part 2 in the flow direction of the bile (fluid) flowing through the bile duct. Specifically, the tubular indwelling device 1 is configured to include a framework part 10 and a membrane part 20.

### <Framework Part>

The framework part 10 is configured to be self-expandable, and in this example, and the framework part 10 has a framework body part 11 having a tubular structure for defining a flow path through which a fluid such as bile passes, and a pair of extending parts 12 arranged so as to extend from a tube end part 11a of the framework body part 11. As illustrated by the dashed line in the figure, the tube end part 11a also corresponds to a boundary for dividing between the framework body part 11 and the pair of extending parts 12 and 12.

A plurality of zigzag annular parts configured such that metal wire rods annularly extend in a circumferential direction while reciprocating in a zigzag shape in the tube axis direction are arranged in the tube axis direction on the framework body part 11. In addition, the framework body part 11 is configured such that adjacent zigzag annular parts are connected to each other in the tube axis direction by metal wire rods at a plurality of positions in the circumferential direction. The framework body part 11 has a cylindrical shape as a whole.

The pair of extending parts 12 and 12 are composed of the metal wire rods, and configured to extend toward the distal end side in the tube axis direction on the width-direction both sides of the framework body part 11. That means, the pair of extending parts 12 and 12 are arranged opposite to each other so as to sandwich the tube axis of the tubular indwelling device 1. The pair of extending parts 12 and 12 are connected to a predetermined position of the framework body part 11 through e.g. a connection part 12a, and configured such that their vertical widths gradually decrease as a distance from the framework body part 11 increases. In addition, each of the pair of extending parts 12 and 12 is formed so as to have a V-shaped part 12c extending diagonally upward and diagonally downward from an apex 12b positioned on the most distal end side in the tube axis direction to the proximal end side in the tube axis direction. As will be described later, the pair of extending parts 12 and 12 functionally serve as support members for supporting a projection part 22 (described later) of the membrane part 20. The pair of extending parts 12 and 12 leave away from each other, and thereby a force for widening the projection part 22 in the width direction may or may not be applied to the projection part 22.

The framework part 10 is configured so as to be expandable and contractable from a diameter-decreased state where the framework part 10 is contracted inward in the radial direction to a diameter-increased state where the framework part 10 is expanded outward in the radial direction. When the framework part 10 is in the diameter-increased state, the tubular indwelling device 1 defines a cylindrical flow path inside the framework part 10. For example, the framework part 10 is configured so as to extend in the tube axis direction while contracting inward in the radial direction by being pulled in the tube axis direction, and shorten in the tube axis direction while expanding outward in the radial direction by being released from the diameter-decreased state. The framework part 10 is configured in such a manner, so that an outer peripheral face of the framework part 10, particularly an outer peripheral face of the framework body part 11 can press an inner face of the bile duct lesion site outward in the radial direction to widen the bile duct lesion site outward in the radial direction, during placement of the tubular indwelling device 1 on the bile duct.

Examples of a material constituting the framework part 10 include known metals or metal alloys typified by a stainless steel, an Ni—Ti alloy (i.e. Nitinol), a titanium alloy, and the like. In addition, a part or the whole of the framework part 10 may be made of an X-ray detectable alloy material such that the position of the framework part 10 can be confirmed from the outside of the body. The framework part 10 may be made of a material other than metal materials such as a ceramic or a resin.

The material, a wire type (e.g. a circular wire rod such as a wire, or an angular wire rod obtained by laser processing), a wire diameter (sectional area), a zigzag reciprocation frequency and a zigzag shape in the circumferential direction, an interval of the wire rods in the tube axis direction (amount of the framework per a unit length), and the like of the metal wire rods constituting the framework part 10 can be appropriately selected depending on the living body lumen where the tubular indwelling device 1 is placed.

### <Membrane Part>

The membrane part 20 is composed of film bodies, and has a configuration in which a cylindrical part 21 arranged along the framework body part 11 of the framework part 10 and the projection part 22 protruding from an end part of the cylindrical part 21 are integrally connected.

The cylindrical part 21 is arranged along the framework body part 11. When the framework body part 11 is expanded during placement of the tubular indwelling device 1 on the bile duct, the cylindrical part 21 defines a flow path for guiding the bile toward the projection part 22. That means, the cylindrical part 21 constitutes the tubular body part 2 together with the framework body part 11. Herein, the cylindrical part 21 may be disposed on the outer peripheral face and the inner peripheral face of the framework body part 11 so as to sandwich the framework body part 11. Alternatively, the cylindrical part 21 may be disposed only on the outer peripheral face or the inner peripheral face of the framework body part 11. The cylindrical part 21 can be fixed to the framework body part 11 e.g. by using a known procedure such as sewing and dipping.

In the membrane part 20, the projection part 22 continuously protrudes from the distal end part of the cylindrical part 21 toward the distal end side in the tube axial direction. The projection part 22 is a portion that discharges the bile to the duodenum during placement of the indwelling device 1 on the bile duct. As a whole, the projection part 22 has a tapered shape in which a flow path sectional area on the distal end side away from the cylindrical part 21 is smaller than a flow path sectional area on the proximal end side connected to the cylindrical part 21. More specifically, in this example, the projection part 22 has a first part 22a and a second part (distal end part-constituting part) 22b. In the first part 22a, the flow path sectional area gradually decreases from the proximal end side to the distal end side along the pair of extending parts 12 and 12 of the framework part 10. The second part 22b extends such that the flow path sectional area is substantially uniform from the first part 22a to the distal end side. The second part 22b is formed in a flat shape such that the film bodies constituting the projection part 22 are brought into substantially close contact with each other in the vertical direction. In addition, the framework part 10 is not provided on the second part 22b, and the membrane part 20 having such a shape is formed by using a known method such as dipping.

An opening on the tube axis direction-distal end part of the projection part 22 (downstream side in the flow direction of the bile) functionally serves as the outflow port 23 for causing the liquid such as bile that has flowed into the projection part 22 from the cylindrical part 21 to flow out into the duodenum. When the liquid does not flow through the tubular indwelling device 1, the outflow port 23 linearly extends in the width direction and is maintained in a closed state. On the other hand, when the liquid flows through the tubular indwelling device 1, the outflow port 23 opens in the vertical direction by a pressure of the liquid itself. As a result, the projection part 22 functionally serves as a check valve for suppressing the outflow of the bile from the bile duct to the duodenum and the reverse flow of foreign substances from the duodenum to the bile duct during the placement of the indwelling device 1 on the bile duct. That means, the valve part 3 is provided on the downstream end part 2a in the bile flow direction in the body part 2 (framework body part 11, cylindrical part 21). In addition, the valve part 3 is formed in a shape tapered in a direction leaving away from the axial-direction mid side of the body part 2, and the outflow port 23 is provided on the distal end part of the valve part 3.

Herein, the “closing” of the outflow port 23 means that the projection part 22 is deformed such that an opening area of the outflow port 23 is decreased. Specifically, the projection part 22 may be deformed to such an extent that the opening area of the outflow port 23 becomes substantially zero. Alternatively, the projection part 22 may be deformed to such an extent that the opening area becomes a predetermined opening area smaller than the opening area during the outflow of bile from the outflow port 23 and larger than zero.

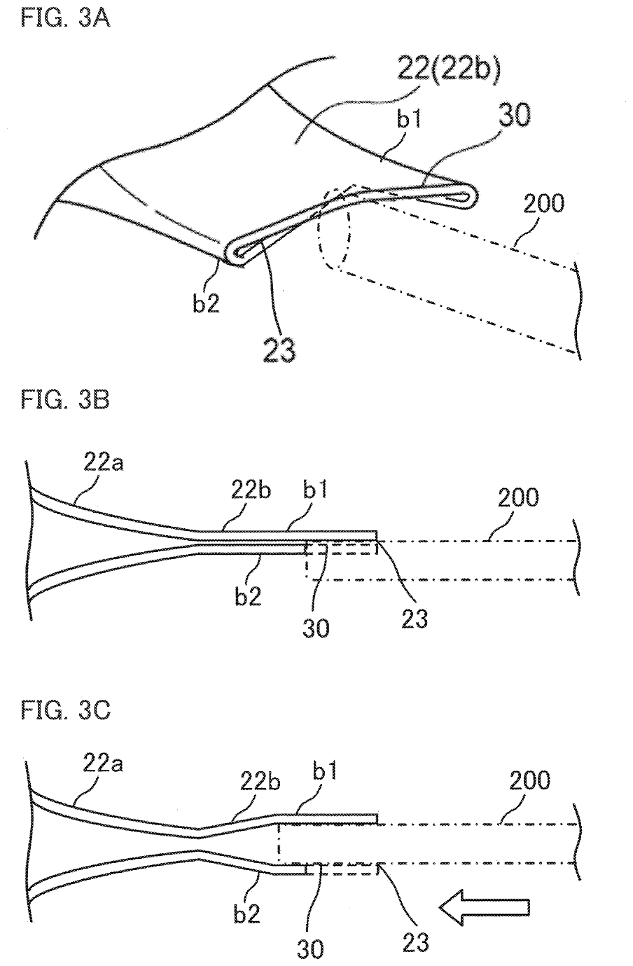

In addition, the valve part 3 includes e.g. a guide part 30 for guiding insertion of a jig 200 (see FIG. 3 (a) and the like) of an endoscope, a treatment device, or the like into the outflow port 23. The guide part 30 is formed on an opening edge part of the outflow port 23 in the membrane part 20, and is composed of e.g. the second part 22b of the projection part 22. Specifically, the guide part 30 is formed a second plate-shaped part b2 of first and second plate-shaped parts b1 and b2 constituting the second part 22b. In addition, the guide part 30 has a shape notched into a predetermined shape (e.g. “V” shape or the like) from the distal end side of the second plate-shaped part b2 to the axial-direction mid side (upstream side in the flow direction). The shape of the guide part 30 is merely an example and the present invention is not limited to this shape. A length, a depth, and the like of oblique sides constituting the notch shape of the guide part 30 can be arbitrarily changed as appropriate. Also, the notch shape is not limited to the “V” shape, and can be arbitrarily changed into e.g. an arc shape or the like, as appropriate.

Examples of a material constituting the membrane part 20 include a fluororesin such as a silicone resin and PTFE (polytetrafluoroethylene), a polyethylene resin such as polyethylene terephthalate, and the like.

Next, insertion of the jig 200 into the valve part 3 of the tubular indwelling device 1 will be explained with reference to FIG. 3 (a) to FIG. 3 (c). FIG. 3 (a) is a perspective view illustrating the state before the insertion of the jig 200 into the valve part 3, FIG. 3 (b) is a sectional view also schematically illustrating the state before the insertion of the jig 200 into the valve part 3. FIG. 3 (c) is a sectional view schematically illustrating the state after the insertion of the jig 200 into the valve part 3. In both FIG. 3 (b) and FIG. 3 (c), a hatched illustration is omitted.

As illustrated in FIG. 3 (a) and FIG. 3 (b), first, a distal end part of the jig 200 is disposed inside the guide part 30 having the notch formed on the second plate-shaped part b2. Herein, the distal end part of the jig 200 is disposed directly below the guide part 30 and is displaced from the lower side (second plate-shaped part b2 side) to the upper side (first plate-shaped part b1 side). Thereby, the jig 200 can be easily disposed inside the guide part 30. Subsequently, as illustrated in FIG. 3 (c), the distal end part of the jig 200 is displaced to a back side of the valve part 3 (in the direction of the white arrow) while pressing the first plate-shaped part b1 upward against a lower face of the first plate-shaped part b1, so that the jig 200 is inserted into the valve part 3. Although not illustrated, the distal end part of the jig 200 is further displaced in the direction of the white arrow, so that the jig 200 is inserted inside the tubular indwelling device 1.

As described above, the tubular indwelling device 1 according to the present embodiment is placed in the living body lumen (bile duct) to define the tubular flow path, and includes the tubular body part 2, and the valve part 3 provided on the downstream end part 2a of the body part 2 in the flow direction of the fluid (bile) flowing through the living body lumen and having the outflow port 23 through which the fluid flows out. The valve part 3 further has a guide part 30 for guiding the insertion of the jig 200 into the outflow port 23. Thus, even when the outflow port 23 of the tubular indwelling device 1 having the valve function with the reverse flow-suppressing effect is closed, the insertion of the jig 200 into the outflow port 23 can be guided by the guide part 30 provided on the valve part 3. The jig 200 can be easily inserted into the inside of the tubular indwelling device 1 having the valve part 3.

In addition, the guide part 30 is formed on the opening edge part of the outflow port 23 on the film body. Thus, the insertion of the jig 200 into the outflow port 23 can be easily guided only by disposing the jig 200 on the guide part 30. Specifically, the valve part 3 is formed in a shape tapered in a direction leaving away from the axial-direction mid side of the body part 2, and the outflow port 23 is provided on the distal end part of the valve part 3. The guide part 30 is composed of a distal end part-constituting part (second plate-shaped part b2) constituting the distal end part on the film body. Thus, the insertion of the jig 200 into the outflow port 23 can be easily guided only by disposing the jig 200 on the guide part 30 formed on the distal end part-constituting part of the tapered valve part 3. In addition, the guide part 30 has a shape notched from the distal end side to the axial-direction mid side of the second plate-shaped part b2. Thereby, the distal end part of the jig 200 can be easily disposed inside the guide part 30 having the notched second plate-shaped part b2, and the jig 200 can be easily inserted into the outflow port 23 while being guided by the guide part 30.

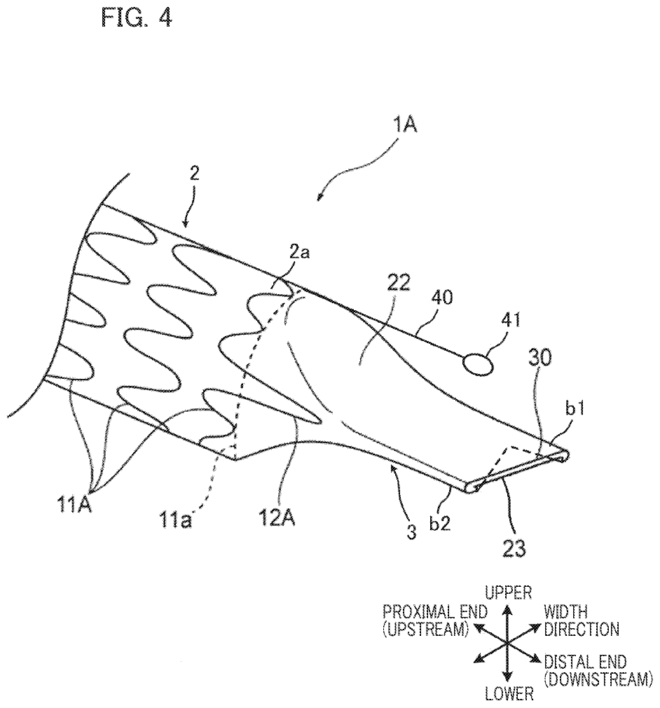

As described above, the present invention has been specifically explained on the basis of the embodiment, but the present invention is not limited to the above embodiment, and can be modified without departing from the gist of the present invention. For example, in the above embodiment, the structure in which the framework body part 11 of the framework part 10 is disposed such that the plurality of zigzag annular parts are arranged in the tube axis direction, has been described as an example. However, the above embodiment is merely an example and is not limited to this structure, and can be arbitrarily modified as appropriate. Specifically, for example, as illustrated in FIG. 4, a framework body part 11A of a tubular indwelling device 1A may be configured such that the metal wire rod spirally pivots while reciprocating in a zigzag shape in the tube axis direction. Additionally, in this case, a pair of extending parts 12A and 12A may be configured such that a part of the metal wire rod pivoting in this manner extends toward the outflow port 23. Also in this case, the pair of extending parts 12A and 12A leave away from each other, and thereby a force for widening the projection part 22 in the width direction may or may not be applied to the projection part 22.

The tubular indwelling device 1A includes a removal auxiliary part 40 to be used in removing the tubular indwelling device 1A after the placement. As a result, for example, even if the tubular indwelling device 1A should be removed after an indwelling state of the tubular indwelling device 1A and a part behind the tubular indwelling device 1A in the living body lumen are confirmed using an endoscope, the tubular indwelling device 1A can be properly removed by engaging a recovery hooking device (not illustrated) positioned on a distal end of a recovery catheter with an engaging part 41 positioned on a distal end of the removal auxiliary part 40.

Additionally, in the above embodiment, the guide part 30 in which the notched shape is formed on the second plate-shaped part b2 constituting the second part 22b, has been described as an example. However, the above embodiment is merely an example and is not limited to this configuration. For example, the notched shape may be formed on the first plate-shaped part b1 constituting the second part 22b. That means, the guide part 30 only needs to be formed on at least one of the first and second plate-shaped parts b1 or b2 constituting the second part (distal end part-constituting part) 22b. Furthermore, the guide part 30 formed in the shape notched into a predetermined shape (e.g. “V” shape or the like) has been described as an example, but the guide part 30 is not necessarily formed in the notched shape. For example, the guide part 30 may have a configuration in which the insertion of the jig into the outflow port 23 is facilitated by differentiating the material, the stretchability, the flexibility, the hardness, and the like between the first and second plate-shaped parts b1 and b2 constituting the second part 22b. In addition, the shape of the guide part 30 is merely an example, the present invention is not limited to this shape, and the shape can be arbitrarily changed as appropriate. For example, the guide part 30 may have a shape in which the distal end part of at least one of the first and second plate-shaped parts b1 and b2 constituting the second part 22b protrudes in a predetermined shape (e.g. a mountain shape (inverted “V” shape) or the like) so as to leave away from the other part.

Additionally, in the above embodiment, the tubular indwelling device 1 that is placed in the bile duct for use has been described as an example, but this case is merely an example, and the present invention is not limited to this case. The tubular indwelling device 1 may be used for another living body lumen for which the valve function with the reverse flow-suppressing effect should be more properly exerted, or another living body lumen for which such a valve function is not required.

Note that the embodiment disclosed in this specification should be regarded as an example in all regards and considered to be unrestrictive. The scope of the present invention is stipulated not by the above explanation but by claims, and intended to include meanings equivalent to claims, and all modifications within the scope of claims.

### DESCRIPTION OF REFERENCE NUMERALS

1, 1A Tubular indwelling device
2 Body part
2a Downstream end part
3 Valve part
10 Framework part
11, 11A Framework body part
12, 12A Extending part
20 Membrane part
21 Cylindrical part
22 Projection part
22b Second part
b2 Second plate-shaped part (distal end part-constituting part)
23 Outflow port
30 Guide part
200 Jig

1. A tubular indwelling device which is placed in a living body lumen to define a tubular flow path, and comprises
a tubular body part, and
a valve part provided on a downstream end part of the tubular body part in a flow direction of a fluid flowing through the living body lumen and having an outflow port from which the fluid flows out, wherein
the valve part further has a guide part for guiding insertion of a jig into the outflow port.

2. The tubular indwelling device according to claim 1, wherein
the outflow port is formed from a film body, and
the guide part is formed on an opening edge part of the outflow port on the film body.

3. The tubular indwelling device according to claim 1 or 2, wherein
the outflow port is formed from a film body,
the valve part is formed in a shape tapered in a direction leaving away from an axial-direction mid side of the tubular body part, and the outflow port is provided on a distal end part of the valve part, and
the guide part is composed of a distal end part-constituting part constituting the distal end part on the film body.

4. The tubular indwelling device according to claim 3, wherein
the distal end part-constituting part is formed in a flat shape such that inner faces of the film body are brought into close contact with each other, and
the guide part is formed on at least one of first and second plate-shaped parts constituting the distal end part-constituting part.

5. The tubular indwelling device according to claim 4, wherein the guide part is formed on at least one of the first and second plate-shaped parts and has a shape notched from a distal end side to an axial-direction mid side.
